(19) United States
(12) Patent Application Publication
(10) Pub. No.: US 2022/0292522 A1
(43) Pub. Date: Sep. 15, 2022

(54) SYSTEM AND METHOD FOR MANAGING AND AUTOMATING COMPLIANCE REQUIREMENTS FOR A BUSINESS ENTITY

(71) Applicant: Ritu Raj Tiwari, Foster City, CA (US)

(72) Inventor: Ritu Raj Tiwari, Foster City, CA (US)

(21) Appl. No.: 17/635,611

(22) PCT Filed: Apr. 9, 2021
(86) PCT No.: PCT/US21/26689

§ 371 Date: Feb. 15, 2022

Related U.S. Application Data
(60) Provisional application No. 63/007,592, filed on Apr. 9, 2020.

(51) Int. Cl. G06Q 30/00; G16H 10/60

(52) U.S. Cl. CPC G06Q 30/018; G16H 10/60

(57) ABSTRACT

A system and method for facilitating and automating regulatory compliance requirements for business entities across various industries. The system includes a secure cloud assembly for the business entity that contains confidential and private information belonging to the entity. A compliance agent in the assembly has external communications with an external compliance database and an external compliance processing server. One or more user workstations has access to confidential and private information contained within the secure cloud assembly. The workstation also has access to the compliance processing server and a communication application for review and meeting compliance requirements. The system automatically monitors and alerts a user to a compliance agenda and requirements, we well as, generates meeting minutes and compliance reports. The method utilizes the described system.

### RELATED APPLICATIONS

This application claims the benefit of U.S. Provisional Application No. 63/007,592, filed on Apr. 9, 2020.

### BACKGROUND OF THE INVENTION

The present invention is directed to a system and method for simplifying and automating the recurring requirements of compliance with third party audits, particularly security compliance management. Compliance with third party audit requirements include HIPPA, HITRUST, SOC2, PCI, and other similar entities. Such requirements include dozens of recurring meetings, multiple committees, meticulous minute taking and action items, and a historical record of compliance with each of these requirements to pass audits and show maturity.

Compliance meetings must meet certain criteria, including scheduling, preparation, attendance time, and record keeping. Scheduling for such meetings has frequency requirements, whether daily, weekly, monthly, quarterly, or annually. Preparation for such meetings requires that documents and items be collected, prepared, reviewed and presented. Attendance time requires that people actually spend the time required to prepare, participate, and attend the meeting. Such participation and attendance includes real-time involvement and offering of commentary. Record keeping for such meetings requires that presentations and commentary be recorded and that follow-up action items be assigned.

The burden of compliance with such requirements is designed primarily for larger organizations that have appropriate infrastructure and staffing. The requirements assume large, dedicated teams that manage overhead for both execution and reporting. Such requirements pose a scalability challenge for smaller organizations. Smaller organizations and nimble startups generally have a tough choice of staying competitive or staying compliant.

Accordingly, there is a need for systems and methods that facilitate monitoring and reporting compliance with business regulations, particular for smaller organizations and entities. The present invention fulfills these needs and provides other related advantages.

### SUMMARY OF THE INVENTION

The present invention is directed to a system and method for managing and automating compliance requirements for a business entity. The system includes a cloud storage assembly containing confidential information and private information in possession of the business entity and a compliance connection agent configured for receiving and processing raw logs and data comprising the confidential information and private information. The cloud storage assembly is protected by a confidential data boundary. A compliance database is remote from the cloud storage assembly but communicatingly connected to the compliance connection agent. The compliance database is configured to transmit confidential information but not private information to the compliance connection agent through the confidential data boundary. A compliance processing server is also remote from the cloud storage assembly but communicatingly connected to the compliance connection agent. The compliance processing server is configured to receive and process confidential information but not private information from the compliance connection agent through the confidential data boundary.

An end user platform or workstation is separate from the cloud storage assembly and communicatingly connected to the cloud storage assembly. The end user platform is configured for receipt and review access to confidential information and private information in the cloud storage assembly through the confidential data boundary. The end user platform is also communicatingly connected to the compliance processing server and configured for review of the received and processed confidential information. A meeting and communication application is communicatingly connected to the compliance processing server and the end user platform. The meeting and communication application is configured for facilitating review of events, discussion of compliance, and preparation of compliance reports.

The confidential information may be contained in data stores, computer systems, and documents stores. The compliance connection agent receives raw logs and data from both the data stores and the computer systems, and processes the raw logs and data into the document stores. The compliance database preferably contains automation source and configuration settings and is configured to communicate the automation source and configuration settings to the compliance connection agent.

The compliance processing server is configured to receive and process reporting and diagnostic data from the confidential information. The automation source and configuration settings may include compliance schedules and compliance goals of the business entity. The compliance processing server may also contain compliance schedules and compliance goals of the business entity transmitted by the compliance database to the compliance connection agent. The private information may include patient health information and personally identifiable information.

The meeting and communication application may facilitate synchronous and asynchronous meetings and communications. The system preferably automates compliance requirements through the meeting and communication application by collecting and reviewing information, contacting employees of the business entity about compliance requirements, presenting information to the employees, recording the employees responses, and logging meeting minutes.

The method for managing and automating compliance requirements for a business entity begins with providing a cloud storage assembly containing a compliance connection agent, confidential information, and private information in possession of the business entity. The confidential information and the private information is protected in the cloud storage assembly using a confidential data boundary. Automation source and configuration settings regarding the compliance requirements are retrieved from a compliance database to the compliance connection agent through the confidential data boundary. Reviewed materials are prepared in the compliance connection agent by applying the automation source and configuration settings to raw logs and data comprising the confidential information and the private information.

A compliance processing server processes reporting data and diagnostic data including the confidential information but not the private information received from the compliance connection agent through the confidential data boundary. The compliance processing server also produces a compliance agenda for the business entity based on the processed reporting data and diagnostic data. An end user platform or workstation is used to access the reviewed materials including the confidential information and the private information in the cloud storage assembly through the confidential data boundary and the compliance agenda in the compliance processing server. A meeting and communication application coordinates meetings about review of the compliance agenda, discussion of compliance events, attendance at meetings, and preparation of compliance reports.

The confidential information and the private information is contained in data stores, computer systems, and documents stores. The compliance connection agent receives raw logs and data from both the data stores and the computer systems, and processes the raw logs and data into the document stores. The automation source and configuration settings include compliance schedules and compliance goals for the business entity. The compliance agenda is produced based on the compliance schedules and compliance goals for the business entity based on the reporting data and diagnostic data.

The private information includes patient health information and personally identifiable information. The meeting and communication application facilitates synchronous and asynchronous meetings and communications. The method automates compliance requirements through the coordinating step by collecting and reviewing information, contacting employees of the business entity about compliance requirements, presenting information to the employees, recording the employees responses, and logging meeting minutes.

Other features and advantages of the present invention will become apparent from the following more detailed description, taken in conjunction with the accompanying drawings, which illustrate, by way of example, the principles of the invention.

### BRIEF DESCRIPTION OF THE DRAWINGS

The accompanying drawings illustrate the invention. In such drawings:

FIG. 1 is a flow diagram illustrating interconnectivity of systems, data, and users according to the inventive system;

FIG. 2 is flow diagram illustrating how the inventive system facilitates and automates compliance meetings;

FIG. 3 is a flow diagram illustrating, in part, how a user sets up the inventive system for use;

FIG. 4 is a flow diagram illustrating, in part, how a user sets up the inventive system for use;

FIG. 5 is a flow diagram illustrating, in part, how a user sets up the inventive system for use;

FIG. 6 is a flow diagram illustrating, in part, how a user sets up the inventive system for use;

FIG. 7 is a flow diagram illustrating, in part, how a user sets up the inventive system for use;

FIG. 8 is a flow diagram illustrating, in part, how a user sets up the inventive system for use;

FIG. 9 is a screen shot of how a preferred embodiment of a main user dashboard might appear in the inventive system;

FIG. 10 is a screen shot of how a preferred embodiment of an events light might appear in the inventive system;

FIG. 11 is a screen shot of how a preferred embodiment of a detailed listing for a compliance event might appear in the inventive system; and

FIG. 12 is a screen shot of how a preferred embodiment of an audit log for a compliance event meeting might appear in the inventive system.

### DETAILED DESCRIPTION OF THE PREFERRED EMBODIMENTS

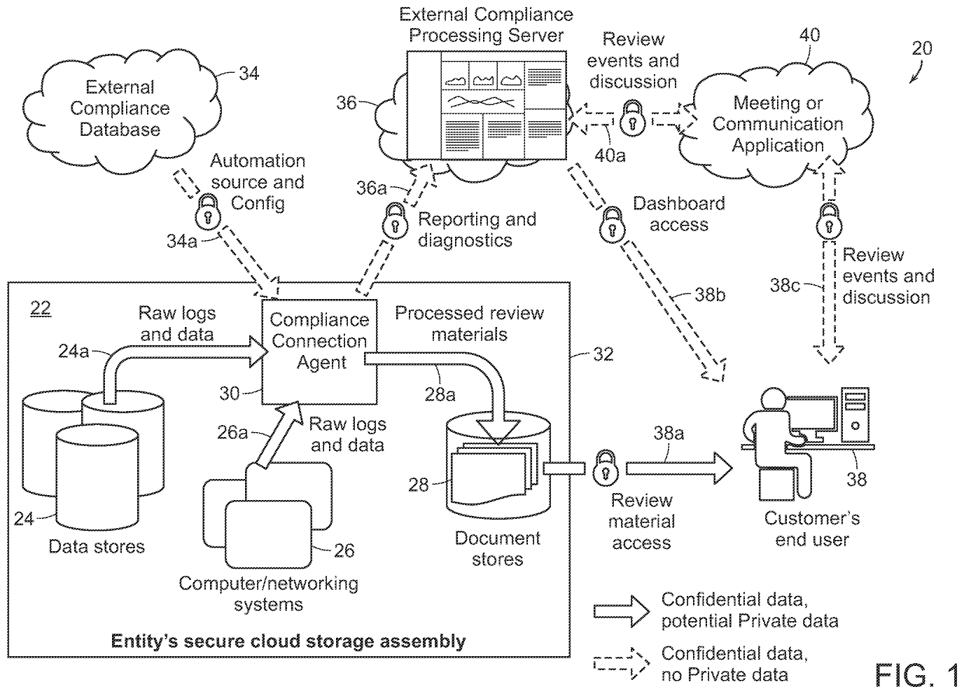

The present invention is directed to a system for facilitating and automating the management and reporting of compliance with regulations, the system generally referred to by reference numeral 20 in FIG. 1. The invention also comprises a method 50 that generally utilizes the features of system 20 to accomplish facilitating and automating the management and reporting of compliance. The inventive automated compliance system 20 and method 50 includes three primary components:

1. Automation tools to collect and prepare review materials;

2. Management of meetings by an automated service providing both synchronous and asynchronous meeting scheduling, presentation of review materials, minute taking, recording and assignment of action items; and

3. Management of minutes and materials stored in the cloud in an audit compliance package.

As shown in FIG. 1, the system 20 is generally organized around a secure cloud storage assembly 22 for a business entity. The storage assembly 22 generally contains data stores 24, a computer or networking system 26, documents stores 28, and a compliance connection agent 30. The compliance connection agent 30 is preferably a computer automated communications hub that directs flow and processing of information and data within and without the storage assembly 22. As needed, the compliance connection agent 30 may be manually controlled to alter or facilitate certain flow and processing of information.

The information and data processed in the storage assembly 22 generally includes confidential information and may potentially contain private information, i.e., patient health information (PHI) and personal identifiable information (PII), depending on the business entity's particular trade or industry. Businesses in the medical fields or financial fields are more likely to include private information in the form of PHI and/or PII.

In the computer automated operation, the compliance connection agent 30 is communicatingly connected to the data stores 24, the computer/networking system 26, and the document stores 28. The compliance connection agent 30 receives both confidential information and private information in raw logs and data 24a, 26a from both the data stores 24 and the computer/networking system 26. The compliance connection agent 30 processes the raw logs and data from the data stores 24 and the computer/networking system 26 to produce processed reviewed materials, containing both confidential information and private information, that are transmitted 28a to the document stores 28.

The storage assembly 22 is preferably contained within a confidential data boundary 32. The confidential data boundary 32 is configured to restrict or entirely prevent communication of confidential information and/or private information outside of the storage assembly 22. Within the confidential data boundary 32, the data stores 24, computer system 26, and their corresponding raw logs and data are only accessible by the compliance connection agent 30. The document stores 28 are only accessible with writing capability by the compliance connection agent 30.

Outside of the storage assembly 22, the system 20 further includes an external compliance database 34, an external compliance processing server 36, and one or more end user workstations 38. The external compliance database 34 contains automation source and configuration information relating to compliance program requirements for business across many and varied business and industries—HIPPA, HITRUST, SOC2, PCI, and many others. The automation source and configuration information generally includes compliance schedules and compliance goals for businesses in each industry.

The compliance connection agent 30 is communicatingly connected to 34a the external compliance database 34 so as to locate and retrieve automation and configuration information for the industry of the business entity that owns the cloud storage assembly 22. The connection 34a is configured to permit the transmission of confidential information as necessary, but not any private information. The confidential data boundary 32 prevents any private information from being included in communication 34a to or from the external compliance database 34.

The compliance connection agent 30 is communicatingly connected to 36a the external compliance processing server 36 so as to transmit reporting and diagnostics information generated as part of its review of the raw data and logs from the data stores 24 and computer system 26. The compliance processing server 36 presents this reporting and diagnostics information for review and described below. The connection 36a is configured to permit the transmission of confidential information as necessary, but not any private information. The confidential data boundary 32 prevents any private information from being included in communication 36a to or from the external compliance processing server 36.

The end user workstations 38 are part of the business entity's operation, but separate from the cloud storage assembly 22, specifically outside of the confidential data boundary 32. The workstations 38 allow employees or contractors of the business entity to access the system 20. Specifically, an end user workstation 38 is communicatingly connected 38a to the document stores 28 to review processed materials therein through the confidential data boundary 32, including both confidential information and private information. The end user workstation 38 cannot modify any of the processed materials contained in the document stores 28.

The end user workstation 38 is also communicatingly connected 38b to the external compliance processing server 36. The connection 38b provides the workstation 38 with review access, i.e., “dashboard” access, to the reporting and diagnostics information provided by the compliance connection agent 30. This connection 38b contains confidential information but does not contain any private information.

The workstations 28 are also communicatingly connected 38c to an external meeting and communication application 40 that facilitates synchronous and asynchronous communications and meetings between multiple employees or contractors or the business entity, preferably to one or more of the workstations 38. From a workstation 38, an employee or contractor can use the application 40 to review events and discussions relating to the reporting and diagnostics information as needed for compliance requirements. This use of the application 40 is accomplished through another communication connection 40a that allows for review, commenting, and discussion of reporting and diagnostics information in the compliance processing server 36. Such communication connections 38c, 40a permit employees and contractors of the business entity to review compliance events and engage in required discussion of the same.

Although outside of the confidential data boundary 32, all communication connections 34a, 36a, 38a, 38b, 38c, and 40a described herein are also appropriately secured or locked against review by computers or networks outside of the system 20. The connection 38a between the workstations 38 and document stores 28 is the only communication outside of the confidential data boundary 32 that may contain private information. Even then, such access is limited to review by authorized workstations 38 and only to the extent any private information may be contained in a document produced from review of the raw logs and data by the compliance connection agent 30.

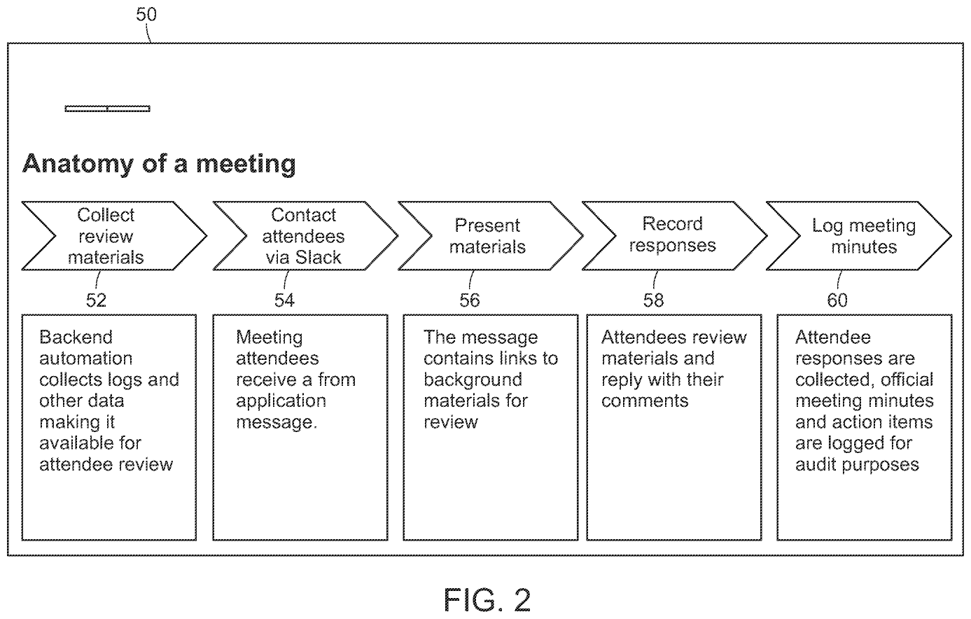

FIG. 2 generally outlines the flow process of a review meeting utilizing the meeting and communication application 40. An automated compliance meeting utilizing the inventive system 20 and method 50 includes collecting and reviewing materials 52, contacting attendees about meeting requirements 54, presenting materials 54, recording attendees' responses 58, and logging the meeting minutes 60. Specifically, in the inventive method 50, a meeting is set up by first collecting review materials 52 as described above with the compliance connection agent 30, the compliance database 34, and the compliance processing server 36. Backend automation from the system 20 collects logs and other data from the user's data stores, making them available for review by attendees.

Required attendees consisting of employees and contractors of the business entity are contacted 54 by message from the application 40. The alert to attendees informs them that the compliance documents are available and ready for review, preferably the message to the required attendees contains links 56 to the compliance materials to be reviewed. Because the application 40 allows for synchronous or asynchronous meetings, attendees can review the materials on their own time and respond with their comments through the meeting application—or simultaneously.

Regardless of synchronous or asynchronous attendance, the application 40 permits attendees at the meeting to record 58 responses or comments following review of the compliance materials. The responses of attendees are collected and official meeting minutes and action items are logged for audit purposes. Action items can be tracked and logged using third-party issue and project tracking software such as JIRA or similar. Once all required attendees have satisfied their compliance requirements, the application 40 creates a log of meeting minutes 60 consisting of attendee responses or comments and other action items that may be logged for audit purposes.

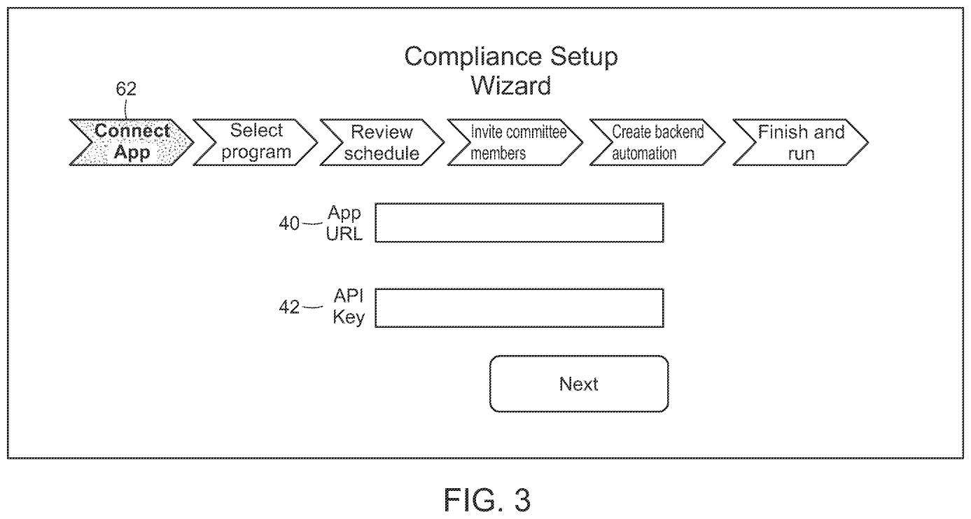

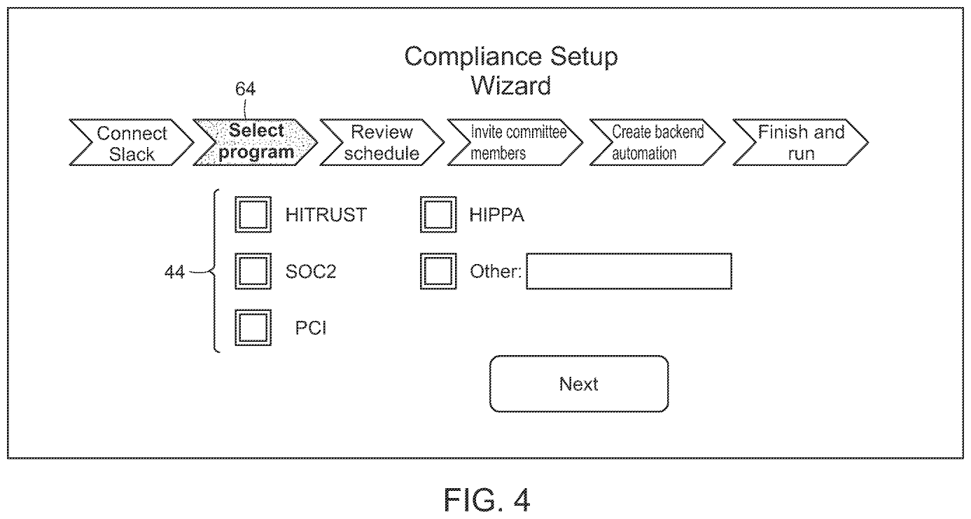

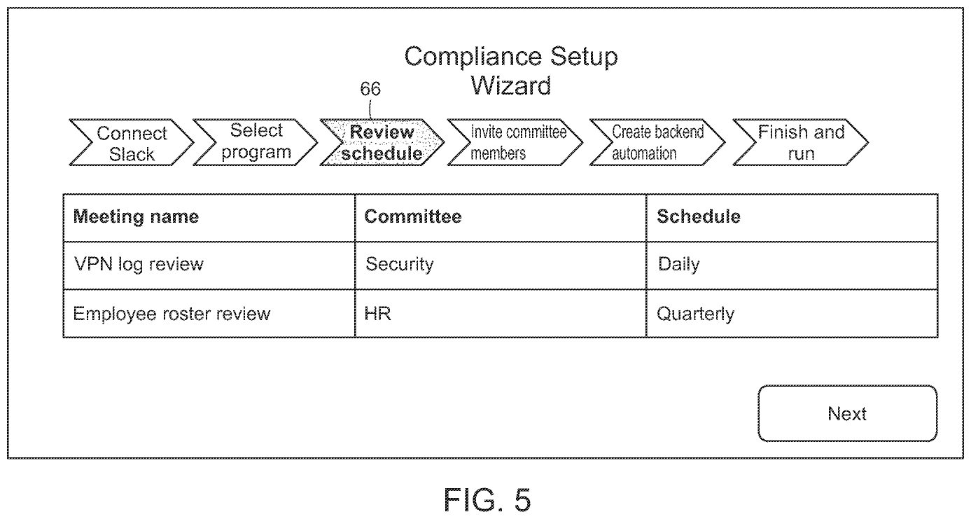

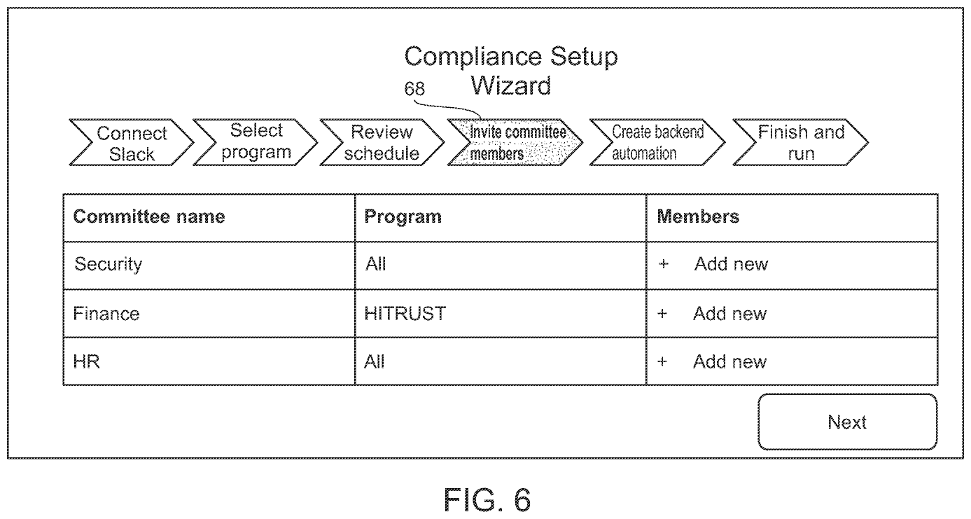

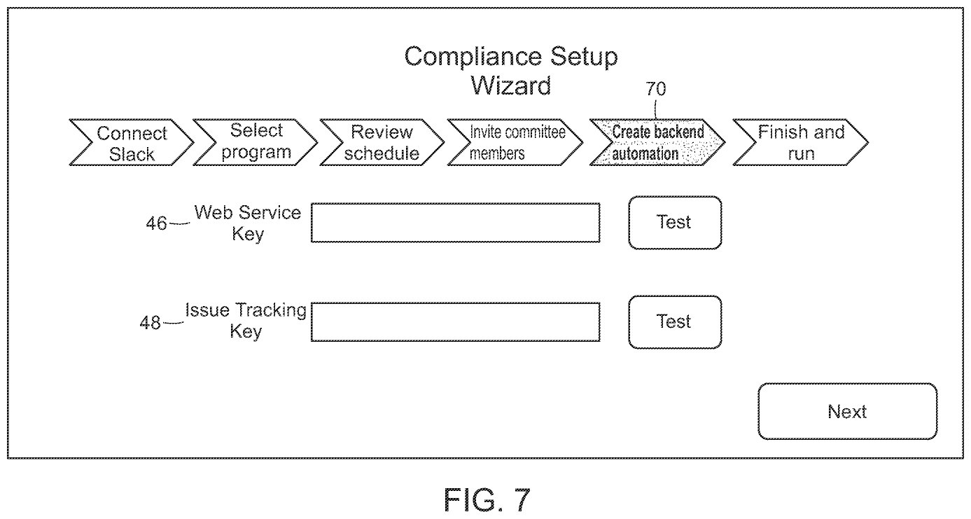

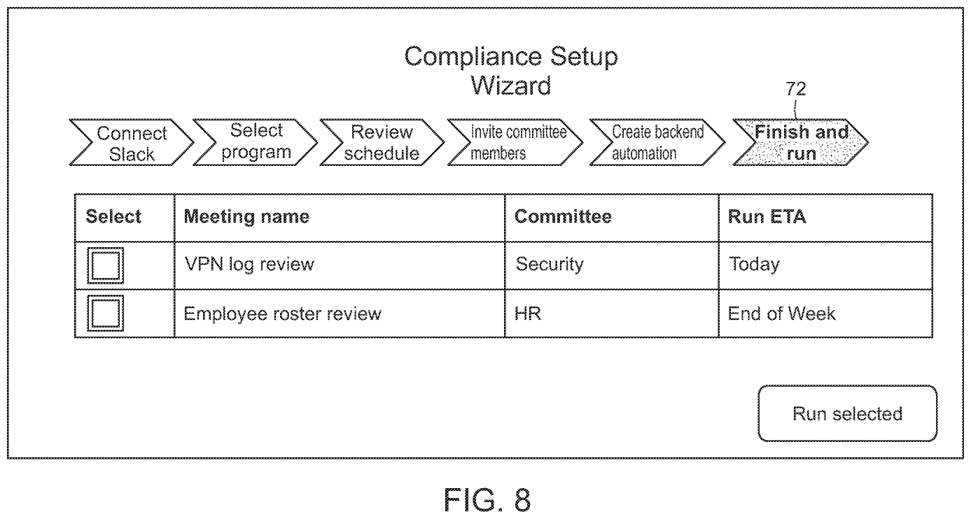

FIGS. 3-8 present screen shots of a typical onboarding process used by a user setting up a compliance connection agent 30 for meeting compliance requirements. Initially, the user sets up communication protocols, including selecting and connecting to a preferred third-party meeting application or communication source 62 for a communication application 40, such as Slack, by providing a meeting universal resource locator (URL). The user also identifies an application programming interface (API) key or other security identifier 42.

The user then identifies particular industry compliance programs or standards 64, such as HITRUST, SOC2, PCI, HIPPA, or others 44. The user then reviews a compliance schedule 66 are prepared by the compliance connection agent 30 described above. The compliance schedule identifies at least the type of compliance meeting, the required attendees, and the meeting frequency. The user then identifies and invites the required committee members 68 based on the compliance schedule. To facilitate the automatic preparation of compliance documents from a user's logs and other data, the user creates backend automation access 70 for the system 20, including security keys and passwords, i.e., a web service key 46 (such as Amazon Web Service) and an issue tracking key 48 (such as JIRA from Atlassian). Then the compliance schedule is finished and run 72, wherein the log of meeting minutes is generated 60.

Once all necessary settings and security accesses are established, the compliance system 20 is finalized and the compliance method 50 is initiated. The compliance system 20 can begin running right away, making certain that a user is in full compliance with all necessary statutory, regulatory, and other industry requirements. As described below, the user dashboard provides a user with an overview of the overall compliance status, including daily, bi-weekly, bi-monthly, quarterly, and other periodic events, as well as the progress being made on each. The compliance status report is tailored to each particular compliance program that the user established during signup.

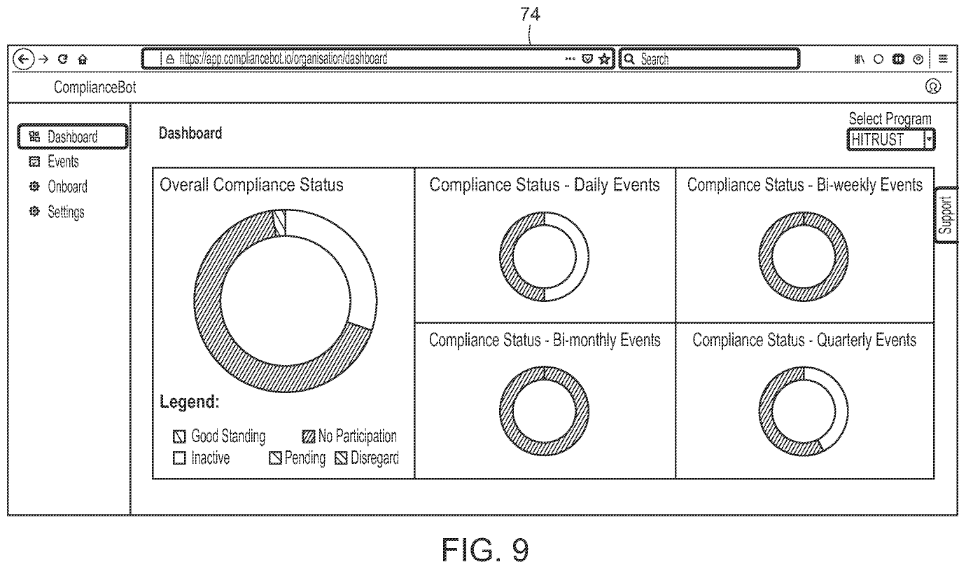

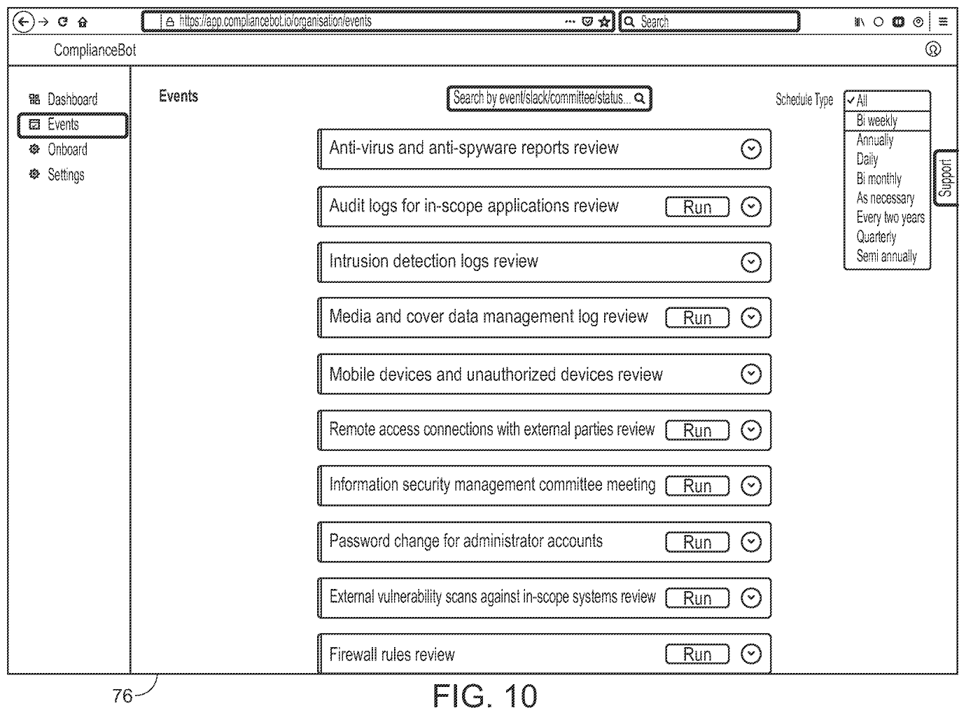

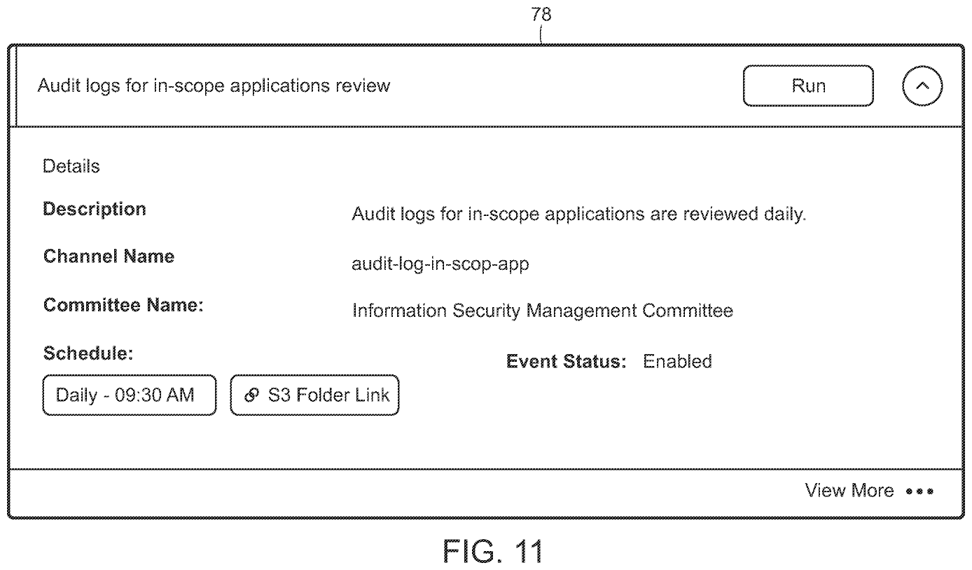

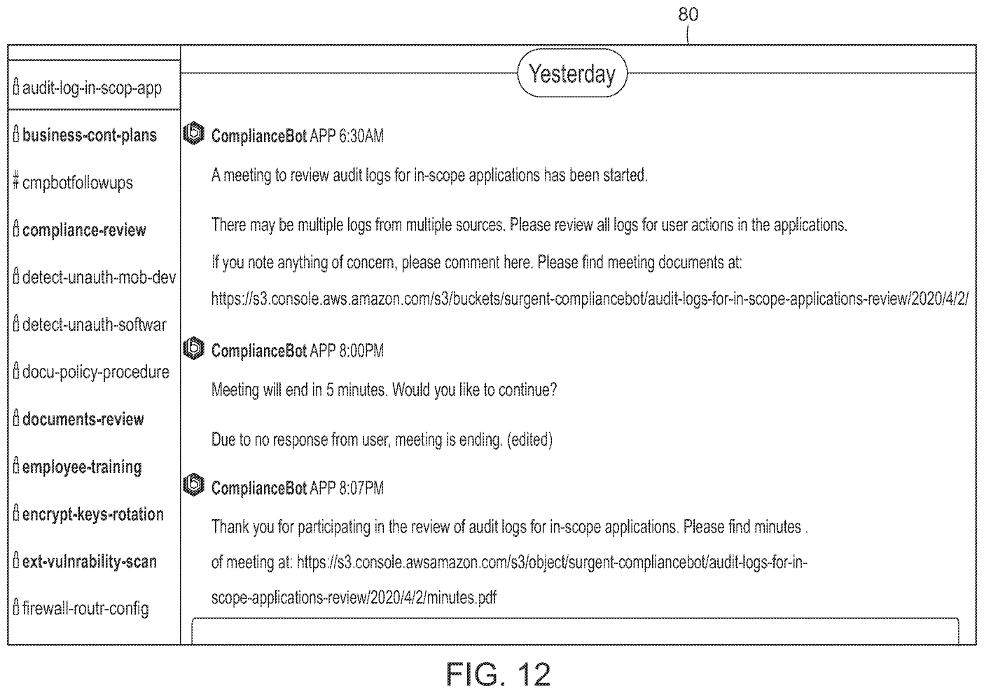

FIG. 9 illustrates a screen displaying a compliance dashboard 74 that a user might review on the compliance processing server 36 from a workstation 38. The dashboard 74 displays overall and individual compliance status updates for a particular entity and program, and includes links to an events list, an onboard report, and a settings page. FIG. 10 illustrates a screen displaying a list of event data 76 regarding meeting agendas and compliance, also from the compliance processing server 36. The list of event data 76 displays each of the various compliance events that a user is required to meet and provides detail and actions for each. FIG. 11 illustrates a detailed listing of a particular event 78 as may be displayed by the compliance processing server 36, including a description of the compliance event, identification of the meeting application, identification of the participants, the status of the event, the schedule for the meetings, and a link to the necessary compliance documents. FIG. 12 illustrates a screen display of how a log of meeting minutes 80 might be recorded for meeting compliance requirements, which may automatically record the document review and meeting occurrences in order to prepare the required minutes for compliance reporting.

The automated compliance system 20 and method 50 can be configured for multiple third-party compliance programs. This configuration may include all available programs or a user may select only those programs that apply to their particular business or industry. Through this configuration, the system 20 automatically generates an organization's compliance schedule. The main user dashboard allows a user to monitor such compliance schedule and other compliance goals.

The confidential data boundary 32 on the system 20 provides data security and protects confidential information, which may include private protected information. Such private protected information might include potential patient health information (PHI) or personally identifiable information (PII) depending upon the specific compliance requirements. A user's data stores 24, computer and networking systems 26, document stores 28, and a system interface or agent 30 to the compliance system 20 are all contained behind the user's secure network boundary 32. The user's system component 22 operates within the secure network boundary 32. It collects raw logs and data required for compliance related reviews from the user's data stores 24 and computer/networking systems 26. The raw logs and data are processed by the system component 22 and review-ready components are placed in the document store 28.

The system component 22 has a secure connection 34a through the user's secure network boundary 32 to receive automation source and configuration settings from an external database 34, such as source control repositories. The system component 22 also has a secure connection 36a to send reporting and diagnostic information through the user's secure network boundary 32 to the compliance system's external processing server 36. The compliance system's external processing server 36 also contains the user's compliance schedule and the main user dashboard.

The external processing server 36 of the compliance system 20 has secure access 40a to the meeting/communication application 40, whether synchronous or asynchronous, to assist with compliance. Such meeting application 40 may be part of the inventive system or a third-party application such as Slack, email, or something similar. The meeting application 40 has a secure bi-directional link 40a to the external processing server 36 to review events and discussion.

From outside the user's secure network boundary 32, a user's employees have secure access to the document store 28 and the processed review materials contained therein through communication line 38a from a workstation 38. The workstation 38 also provides the user's employees with secure access to the compliance system's external processing server 36, specifically the main user dashboard, to review compliance schedule, status, and progress. The workstation 38 also provides the user's employees with secure bi-directional access to the meeting application 40 to review events and participate in meeting discussions. When asynchronous, the user's employees can review documents and participate in the required compliance meetings on different schedules while still satisfying the compliance requirements.

Although particular embodiments have been described in detail for purposes of illustration, various modifications may be made without departing from the scope and spirit of the invention.

What is claimed is:

1. A system for managing and automating compliance requirements for a business entity, comprising:
a cloud storage assembly containing confidential information and private information in possession of the business entity and a compliance connection agent configured for receiving and processing raw logs and data comprising the confidential information and private information, wherein the cloud storage assembly is protected by a confidential data boundary;
a compliance database remote from the cloud storage assembly communicatingly connected to the compliance connection agent, wherein the compliance database is configured to transmit confidential information but not private information to the compliance connection agent through the confidential data boundary;
a compliance processing server remote from the cloud storage assembly communicatingly connected to the compliance connection agent, wherein the compliance processing server is configured to receive and process confidential information but not private information from the compliance connection agent through the confidential data boundary;
an end user platform separate from the cloud storage assembly communicatingly connected to the cloud storage assembly, wherein the end user platform is configured for receipt and review access to confidential information and private information in the cloud storage assembly through the confidential data boundary;
wherein the end user platform is also communicatingly connected to the compliance processing server and configured for review of the received and processed confidential information; and
a meeting and communication application communicatingly connected to the compliance processing server and the end user platform, configured for facilitating review of events, discussion of compliance, and preparation of compliance reports.

2. The system of claim 1, wherein the confidential information comprises data stores, computer systems, and documents stores.

3. The system of claim 2, wherein the compliance connection agent receives raw logs and data from both the data stores and the computer systems, and processes the raw logs and data into the document stores.

4. The system of claim 1, wherein the compliance database contains automation source and configuration settings and is configured to communicate the automation source and configuration settings to the compliance connection agent.

5. The system of claim 1, wherein the compliance processing server is configured to receive and process reporting and diagnostic data from the confidential information.

6. The system of claim 1, wherein the automation source and configuration settings include compliance schedules and compliance goals of the business entity.

7. The system of claim 6, wherein the compliance processing server contains compliance schedules and compliance goals of the business entity transmitted by the compliance database to the compliance connection agent.

8. The system of claim 1, wherein the private information comprises patient health information and personally identifiable information.

9. The system of claim 1, wherein the meeting and communication application facilitates synchronous and asynchronous meetings and communications.

10. The system of claim 1, wherein the system automates compliance requirements through the meeting and communication application by collecting and reviewing information, contacting employees of the business entity about compliance requirements, presenting information to the employees, recording the employees responses, and logging meeting minutes.

11. A method for managing and automating compliance requirements for a business entity, comprising:
providing a cloud storage assembly containing a compliance connection agent, confidential information, and private information in possession of the business entity;
protecting the confidential information and the private information in the cloud storage assembly using a confidential data boundary;
retrieving automation source and configuration settings regarding the compliance requirements from a compliance database to the compliance connection agent through the confidential data boundary;
preparing reviewed materials in the compliance connection agent by applying the automation source and configuration settings to raw logs and data comprising the confidential information and the private information;
processing in a compliance processing server reporting data and diagnostic data comprising the confidential information but not the private information received from the compliance connection agent through the confidential data boundary;
producing in the compliance processing server a compliance agenda for the business entity based on the processed reporting data and diagnostic data;
accessing on an end user platform the reviewed materials comprising the confidential information and the private information in the cloud storage assembly through the confidential data boundary and the compliance agenda in the compliance processing server; and
coordinating through a meeting and communication application about review of the compliance agenda, discussion of compliance events, attendance at meetings, and preparation of compliance reports.

12. The system of claim 11, wherein the confidential information and the private information is contained in data stores, computer systems, and documents stores.

13. The system of claim 12, wherein the compliance connection agent receives raw logs and data from both the data stores and the computer systems, and processes the raw logs and data into the document stores.

14. The system of claim 11, wherein the automation source and configuration settings include compliance schedules and compliance goals for the business entity.

15. The system of claim 14, wherein the compliance agenda is produced based on the compliance schedules and compliance goals for the business entity based on the reporting data and diagnostic data.

16. The system of claim 11, wherein the private information comprises patient health information and personally identifiable information.

17. The system of claim 11, wherein the meeting and communication application facilitates synchronous and asynchronous meetings and communications.

18. The system of claim 11, wherein the method automates compliance requirements through the coordinating step by collecting and reviewing information, contacting employees of the business entity about compliance requirements, presenting information to the employees, recording the employees responses, and logging meeting minutes.
